(19) United States
(12) Patent Application Publication
(10) Pub. No.: US 2026/0275342 A1
(43) Pub. Date: Sep. 17, 2026

(54) PVAX14 NUCLEIC ACID IN COMBINATION WITH ALL-TRANS RETINOIC ACID (ATRA) AND A CHECKPOINT INHIBITOR FOR THE TREATMENT OF CANCER

(71) Applicants: VIVADJU, Paris (FR); INSTITUT NATIONAL DE LA SANTE ET DE LA RECHERCHE MEDICALE, Paris (FR); CENTRE NATIONAL DE LA RECHERCHE SCIENTIFIQUE, Paris (FR); UNIVERSITÉ PARIS CITÉ, Paris (FR); UNIVERSITÉ DE NANTES, Nantes (FR); UNIVERSITÉ D'ANGERS, Angers (FR); ASSISTANCE PUBLIQUE-HOPITAUX DE PARIS, Paris (FR)

(72) Inventors: Rose Ann PADUA, Paris (FR); Christine CHOMIENNE, Bois Le Roi (FR); Marie PLA, Paris (FR); Patricia KRIEF, Gonesse (FR); Bruno PITARD, Nantes (FR)

(21) Appl. No.: 19/469,775

(22) PCT Filed: Mar. 29, 2024
(86) PCT No.: PCT/EP2024/058788

§ 371 Date: Sep. 26, 2025

(30) Foreign Application Priority Data
Mar. 30, 2023 (EP) 23305449.3

(51) Int. Cl. C12N 15/113; A61K 31/203; A61K 31/706; A61K 31/7105; A61K 33/36; A61K 39/00; A61P 35/00; C07K 16/28

(52) U.S. Cl. CPC C12N 15/113; A61K 31/203; A61K 31/706; A61K 31/7105; A61K 33/36; A61P 35/00; C07K 16/2803; C07K 16/2818; C07K 16/2827; A61K 2039/505; C12N 2310/11

(57) ABSTRACT

The present invention relates to a pharmaceutical combination comprising (i) at least one nucleic acid comprising an antisense and flipped sequence of a fragment of a sequence coding for a kanamycin resistance protein, (ii) at least one non-immuno-suppressive inducer of tumor cell apoptosis and (iii) at least one checkpoint inhibitor. Said nucleic acid is preferably pVAX14 plasmid, said non-immunosuppressive inducer of tumor cell apoptosis is preferably ATRA or an arsenic-related compound and said checkpoint inhibitor is preferably an anti-PD1 or anti-CD47 antibody or a fragment thereof. The present invention also relates to the pharmaceutical combination as defined above for use in the treatment of cancer, in particular of breast cancer to slow or stop the development of cancer.

### FIELD OF THE INVENTION

The present invention relates to the treatment of cancer.

### TECHNICAL BACKGROUND

The development of malignancy is a two-edged sword with an expansion of abnormal cells that acquire properties of stem cells and the increasing immunosuppression as disease progresses. Acquisitions of stemness and immuno-inhibition are directly associated with specific changes of gene expression programs associated with epigenetic regulation, which is now considered a bridge between genotype and phenotype integrating both intrinsic (gene mutation) and environmental (crosstalk between cancer cell and biological environment) signals. The microenvironment includes the immune system and how the tumour cells interact with the immune system can be exploited.

The Inventors have shown in mouse models that DNA plasmids containing PML-RARa sequences enhance survival when combined with a differentiating/apoptotic/immunomodulators, such as a vitamin A derivative, all-trans retinoic acid (ATRA). A specific DNA plasmid with PML-RARa sequences has previously been shown in acute promyelocytic leukaemia (APL) mice to enhance survival when combined with ATRA, with a reduction of minimal residual disease (MRD) (Furugaki et al, 2010, Blood 15:653-656; Padua et al, 2003, Nature Medicine 9:1413-1417; Pokorna et al, 2013, Molecular and Cellular Probes 27:1-5). The Inventors developed a DNA plasmid construct, pVAX14, in combination with the immunomodulatory all-trans retinoic acid (ATRA), to activate and enhance the immune system in all patients with cancer, the cancer cell itself having provided the antigen(s) to mount the tumor specific responses. The efficacy of this nonspecific vaccine pVAX14 was found to be similar to that of the specific vaccine in the APL model and significantly different from ATRA alone, p<0.0014) (Le Pogam et al, 2015, Oncotarget 6:32494-32508). Enhanced survival correlated with enhanced immune responses and the control or targeting of disease in the APL mouse model (Furugaki et al, 2010; Padua et al, 2003; Pokorna et al, 2013; Robin et al, 2006, Blood 108:1972-1974; Le Pogam et al, 2015; Patel et al, 2015, Blood Cancer Journal December 11; 5 (12): e374. doi: 10.1038/bcj.2015.102) and similar results were obtained in a mouse model of high risk myelodysplasia (HR-MDS) (Le Pogam et al, 2015, Oncotarget, vol 6, 32, 32494-32508; Omidvar et al, 2007, Cancer Res 67:11657-11667); this correlated with increased immune responses and disease control.

The checkpoint inhibitor anti-PD-1 antibody has been used in many different cancers with some success. However, resistance is an emerging problem (reviewed in Bagchi et al. 2021, Annu Rev Pathol. January 24; 16:223-249. doi: 10.1146/annurev-pathol-042020-042741).

There is still a need for improved treatments of cancer.

### DESCRIPTION OF THE INVENTION

The Inventors have unexpectedly found that the combined use of (i) a nucleic acid comprising an antisense and flipped sequence of a fragment of a sequence coding for the kanamycin resistance protein, such as pVAX14, (ii) a non-immunosuppressive inducer of tumor cell apoptosis, such as ATRA, and (iii) a checkpoint inhibitor, such as a PD1 inhibitor, reduces tumor burden and increases memory T-cells, thereby resulting in a durable protection against cancer.

For example, the combination of the DNA plasmid pVAX14 and ATRA with anti-PD-1 antibody has been used in a transplantable model of breast cancer. These cells have the luciferase gene and can be imaged with the IVIS imager. It was observed a reduction in tumor burden, a reduction in PD-1 levels and an increase in memory T-cells when using this combination. The long-term survivors have been followed up to their lifetime and were challenged with breast cancer cells 4 times over the follow-up period, with no evidence of tumor growth, where naïve control mice all developed tumors.

Advantageously, uptake of the nucleic acid comprising the antisense and flipped sequence was increased when delivered in a nano-vehicle, such as in nanotaxi®.

Both the nucleic acid comprising the antisense and flipped sequence and the check point inhibitor can be formulated with a nano-vehicle, such as nanotaxi®, thereby allowing them to be delivered in the form of a single pharmaceutical composition.

A first object of the inventions is thus a pharmaceutical combination comprising:
(i) at least one nucleic acid comprising an antisense and flipped sequence of a fragment of the sequence coding for a kanamycin resistance protein, wherein said nucleic acid is capable of eliciting an immune response in a mammal,
(ii) at least one non-immunosuppressive inducer of tumor cell apoptosis, and
(iii) at least one checkpoint inhibitor.

Said nucleic acid (i) is preferably formulated with a nano-vehicle.

Said nucleic acid preferably comprises a sequence selected from the group consisting of:
a) sequence SEQ ID NO: 2,
b) a sequence at least 80% identical to SEQ ID NO: 2,
c) a fragment of at least 50 consecutive nucleotides of SEQ ID NO: 2, and
d) sequence SEQ ID NO: 3.

Said antisense and flipped sequence preferably encodes at least one immunogenic peptide selected from the group consisting of sequence SEQ ID NO: 4 and SEQ ID NO: 5.

Said nucleic acid preferably encodes an immunogenic peptide of sequence SEQ ID NO: 6.

Said checkpoint inhibitor is preferably selected from the group consisting of a PD-1 inhibitor, a PD-L1 inhibitor, a CD47 inhibitor and a CTLA-4 inhibitor.

Said non-immunosuppressive inducer of tumor cell apoptosis is preferably ATRA, arsenic, arsenic trioxide or azacytidine.

In the pharmaceutical combination as defined above, each of said nucleic acid (i), said non-immunosuppressive inducer of tumor cell apoptosis (ii) and said checkpoint inhibitor (iii) may be provided in the form of a separate pharmaceutical composition.

In the pharmaceutical combination as defined above, said nucleic acid (i) and said checkpoint inhibitor (iii) may be provided in the same pharmaceutical composition.

In the pharmaceutical combination as defined above, said nucleic acid (i), said non-immunosuppressive inducer of tumor cell apoptosis (ii) and said checkpoint inhibitor (iii) may be provided in the form of one or more doses of a pharmaceutical composition.

Another object of the invention is the pharmaceutical combination as defined above, for use as a medicament, preferably in the treatment of cancer, such as a solid tumor, preferably breast cancer, or a blood cancer.

Said nucleic acid (i), said non-immunosuppressive inducer of tumor cell apoptosis (ii) and/or said checkpoint inhibitor (iii) may be administered simultaneously or sequentially.

Said nucleic acid (i), said non-immunosuppressive inducer of tumor cell apoptosis (ii) and/or said checkpoint inhibitor (iii) may be administered by intradermal, intra-epidermal, subcutaneous, intramuscular, intravenous, oral route or intratumoral route.

The present invention also relates to the pharmaceutical combination for use as defined above, wherein:
said nucleic acid (i) is administered at a dosage of from 500 μg to 2 mg,
said non-immunosuppressive inducer of tumor cell apoptosis (ii) is ATRA and is administered at a dosage of 45 mg/m² and/or
said checkpoint inhibitor (iii) is administered at a dosage of from 1 to 10 mg/kg.

The present invention also relates to the pharmaceutical combination for use as defined above, wherein:
said nucleic acid (i) is administered once or at least twice, preferably with an interval of two weeks between two administrations,
said non-immunosuppressive inducer of tumor cell apoptosis (ii) is preferably ATRA and is administered once a day for 10 days, each time said nucleic acid (i) is administered, and/or
said checkpoint inhibitor (iii) is administered each time said nucleic acid (i) is administered.

Nucleic acid comprising an antisense and flipped sequence of a fragment of the sequence coding for the kanamycin resistance protein

The pharmaceutical combination according to the invention comprises at least one nucleic acid comprising an antisense and flipped sequence of a fragment of a sequence coding for the kanamycin resistance protein.

By “antisense and flipped sequence of sequence X”, it is herein meant the reverse complement sequence of sequence X.

This means that said antisense and flipped sequence of a fragment of a sequence coding for the kanamycin resistance protein does not encode a kanamycin resistance protein, nor a fragment thereof.

However, using an antisense and flipped sequence allowed creating novel coding sequences, in particular encoding new immunogenic peptides.

Consequently, the nucleic acid as defined above is capable of eliciting an immune response in a mammal, such as in a mouse, rat or human.

Methods for measuring an immune response are well known in the art. For example, the methods disclosed in Padua et al. (2003, Nature Medicine 9:1413-1417) may be used.

By “nucleic acid”, it is herein meant to the phosphate ester polymeric form of ribonucleosides (“RNA”) or deoxyribonucleosides (“DNA”), or any phosphoester analogs thereof, such as phosphorothioates and thioesters, in particular in either single stranded form or a double-stranded helix.

The nucleic acid as defined above is preferably a double-stranded DNA.

The nucleic acid as defined above may be a linear or circular DNA.

The nucleic acid as defined above is preferably an isolated nucleic acid.

By the term “isolated”, it is herein particularly meant a compound, which is isolated from a human or animal body, from a cell and/or from a library of compounds.

As used herein, the term “kanamycin resistance protein” refers to an enzyme capable of conferring resistance to kanamycin.

Preferably, the sequence coding for the kanamycin resistance protein consists of nucleotides 1226 to 2020 of sequence SEQ ID NO: 7.

The nucleic acid as defined above may further comprise a reverse complement sequence of a fragment of pVAX1 plasmid, in particular a reverse complement sequence of a fragment of pVAX1 plasmid which is adjacent to the sequence coding for the kanamycin resistance protein.

10 The sequence of the pVAX1 plasmid is shown as SEQ ID NO: 7.

Said fragment of the sequence coding for the kanamycin resistance protein may be of any length, e.g. of at least, at most or about 50, 100, 150, 200, 250, 300, 350, 400, 450, 473 or 500 consecutive nucleotides.

Said fragment for example consists of from 50 to 500 consecutive nucleotides, preferably from 100 to 500 consecutive nucleotides, for example from 100 to 473 consecutive nucleotides.

The antisense and flipped sequence as defined above preferably comprises a 20 sequence selected from the group consisting of:
a) a reverse complement sequence of a fragment of SEQ ID NO: 7, wherein said fragment comprises at least 50 consecutive nucleotides of the nucleotides located from position 1226 to position 2020 of SEQ ID NO: 7;
b) sequence SEQ ID NO: 1;
c) a sequence at least 80%, preferably at least 85%, at least 90%, at least 95%, at least 96%, at least 97%, at least 98% or at least 99% identical to SEQ ID NO: 1;
d) a fragment of at least 50, preferably at least 100, at least 150, at least 200, at least 250, at least 300, at least 350, at least 400 or at least 450, consecutive nucleotides of SEQ ID NO: 1; and
e) a derivative of (a) or (b) capable of eliciting an immune response in a mammal.

The antisense and flipped sequence as defined above may for example comprise or consist of the reverse complement sequence of a fragment of SEQ ID NO: 7, wherein said fragment of SEQ ID NO: 7 comprises at least, at most or about 50, 100, 150, 200, 250, 300, 350, 400, 450, 473 or 500 consecutive nucleotides of the nucleotides located from position 1226 to position 2020 of SEQ ID NO: 7.

The fragment of SEQ ID NO: 7 may for example comprise or consist of 50 to 500, 100 to 473, 100 to 500, 200 to 500, 300 to 500, 400 to 500 or 450 to 500 consecutive nucleotides of the nucleotides located from position 1226 to position 2020 of SEQ ID NO: 7.

The antisense and flipped sequence as defined above preferably encodes at least one immunogenic peptide selected from the group consisting of sequence SEQ ID NO: 4 and SEQ ID NO: 5.

The immunogenic peptide of sequence SEQ ID NO: 4 corresponds to the peptide encoded by ORF1 starting at position 813 in sequence SEQ ID NO: 3,

The immunogenic peptide of sequence SEQ ID NO: 5 corresponds to the peptide encoded by ORF2 starting at position 818 in sequence SEQ ID NO: 3.

The nucleic acid as defined above preferably comprises a sequence selected from the group consisting of:
a) sequence SEQ ID NO: 2;
b) a sequence at least 80%, preferably at least 85%, at least 90%, at least 95%, at least 96%, at least 97%, at least 98% or at least 99% identical to SEQ ID NO: 2;
c) a fragment of at least 50, preferably at least 100, at least 150, at least 200, at least 250, at least 300, at least 350, at least 400 or at least 450, consecutive nucleotides of SEQ ID NO: 2; and
d) a derivative of SEQ ID NO: 2 capable of eliciting an immune response in a mammal.

The nucleic acid as defined above preferably encodes an immunogenic peptide of sequence SEQ ID NO: 6.

The immunogenic peptide of sequence SEQ ID NO: 6 corresponds to the peptide encoded by ORF5 starting at position 1432 of sequence SEQ ID NO: 3.

The nucleic acid as defined above may further comprise a sequence that encodes a tumor antigen.

In another embodiment, the nucleic acid as defined above does not comprise any sequence that encodes a tumor antigen.

By the expression “nucleic acid having a sequence at least x % identical to sequence Y”, it is herein meant that the sequence of the nucleic acid is identical to sequence Y except that the sequence may include up to 100-x nucleotide alterations per each 100 nucleotides of the query sequence. In other words, to obtain a nucleic acid having a sequence at least x % identical to a query sequence, up to 100-x % of the nucleotides of the sequence may be inserted, deleted, or substituted with another nucleotide. “x” is preferably comprised between 80 and 100.

The term “derivative” includes homologues, mutants and naturally-occurring variants such as allelic variants, splice variants or variants obtained through proteolytic processing. Derivatives consisting of an amino acid sequence at least x % identical to a reference sequence may comprise mutation(s), such as deletion(s), insertion(s) and/or substitution(s) compared to the reference sequence. In case of substitutions, the derivative consisting of an amino acid sequence at least x % identical to a reference sequence may correspond to a homologous sequence derived from another species than the reference sequence. The substitution may correspond to a conservative substitution as indicated in the table below.

| Conservative substitutions | Type of Amino Acid |
|---|---|
| Ala, Val, Leu, Ile, Met, | Amino acids with aliphatic |
| Pro, Phe, Trp | hydrophobic side chains |
| Ser, Tyr, Asn, Gln, Cys | Amino acids with uncharged but |
| | polar side chains |
| Asp, Glu | Amino acids with acidic side chains |
| Lys, Arg, His | Amino acids with basic side chains |
| Gly | Neutral side chain |

The nucleic acid as defined above may be provided in the form of a vector.

The nucleic acid as defined above is preferably a vector, for example a plasmid.

In said vector, the antisense and flipped sequence is preferably placed under the control of signals (e.g. a promoter, a terminator and/or an enhancer) allowing the transcription of the antisense and flipped sequence.

The vector as defined above is preferably a DNA vaccination vector, i.e. a vector specifically designed for the development of a DNA vaccine.

The DNA vaccination vector may correspond to or be derived from, e.g. the pVAX1 of sequence SEQ ID NO: 7 (Invitrogen, Carlsbad, California, USA) or the pCDNA3 (Invitrogen, Carlsbad, California, USA) expression vector.

The vector as defined above may further comprise a resistance gene, for example conferring resistance to an antibiotic, such as ampicillin or kanamycin.

Alternatively, the vector as defined above does not comprise any gene conferring resistance to an antibiotic.

The vector as defined above preferably comprises sequence SEQ ID NO: 2.

The vector as defined above preferably allows the expression of at least one immunogenic peptide selected from the group consisting of sequence SEQ ID NO: 4, SEQ ID NO: 5 and SEQ ID NO: 6.

The vector as defined above preferably allows the expression of immunogenic peptide SEQ ID NO: 4, immunogenic peptide SEQ ID NO: 5 and, optionally, immunogenic peptide SEQ ID NO: 6.

A preferred vector as defined above comprises or consists of sequence SEQ ID NO: 3.

In one preferred embodiment, the nucleic acid as defined above comprises sequence SEQ ID NO: 3, more preferably is the pVAX14 plasmid of sequence SEQ ID NO: 3.

### Non-Immunosuppressive Inducer of Tumor Cell Apoptosis

The pharmaceutical combination according to the invention further comprises at least one non-immunosuppressive inducer of tumor cell apoptosis.

Non-immunosuppressive inducers of tumor cell apoptosis are well-known in the art.

The non-immunosuppressive inducer of tumor cell apoptosis may for example be a retinoid compound, an arsenic-related compound, CD437, a compound activating CD44 (such as an antibody or hyaluronic acid), an hematopoietic growth and differentiation factor, 5-azacytidine, another demethylating agent, farnesyl transferase inhibitors (FTI), histone deacetylate inhibitors (HDACi), a small molecule such as Imatinib, a BH3 mimetic inhibitor, such as ABT 737, ABT-199, or a RAC1 inhibitor.

5-azacytidine is also referred to as azacytidine in the following.

By “retinoid compound”, it is herein meant a vitamin A derivative.

The retinoid compound may be selected from the group consisting of retinoic acid (RA), all-trans retinoic acid (ATRA), 9-cis RA, 4-HPPR, 13-cis RA and a synthetic analog of retinoic acid, such as AM 580.

Preferably, the retinoid compound is ATRA or AM 580.

ATRA is also referred to as tretinoin or all-trans retinoic acid.

ATRA is a derivative of vitamin A and is a ligand for the retinoic acid receptor (RAR).

ATRA is referred under CAS number 302-79-4.

AM 580 is an analog of retinoic acid.

AM 580 is referred under CAS number 102121-60-8.

By “arsenic-related compound”, it is herein meant any compound that, just as arsenic, is a phosphatase inhibitor or is capable of creating covalent bonds by dithiol group binding. The arsenic-related compound may for example be arsenic or arsenic trioxide (As₂O₃).

In a preferred embodiment, the non-immunosuppressive inducer of tumor cell apoptosis is arsenic, arsenic trioxide (ATO), all-trans retinoic acid (ATRA), azacytidine or AM 580.

The non-immunosuppressive inducer of tumor cell apoptosis preferably has adjuvant activity towards the biological response elicited by the nucleic acid comprising the antisense and flipped sequence as defined above.

By “adjuvant activity”, it is herein meant an effect achieved by the combination of two components that is greater than the effect of either of the two components alone.

The non-immunosuppressive inducer of tumor cell apoptosis in the pharmaceutical combination is preferably ATRA or arsenic trioxide.

### Checkpoint Inhibitor

The pharmaceutical combination according to the invention also comprises at least one checkpoint inhibitor.

By “checkpoint inhibitor”, it is herein meant a molecule, which blocks checkpoint proteins.

Checkpoint proteins are regulators of the immune system, which are expressed by some cells of the immune system cells, such as T cells, but also by some cancer cells. Examples of checkpoint proteins include PD-1, PD-L1, CD47 and CTLA-4.

The checkpoint inhibitor as defined above may for example be selected from the group consisting of a PD-1 inhibitor, a PD-L1 inhibitor, a CD47 inhibitor and a CTLA-4 inhibitor.

The checkpoint inhibitor as defined above is preferably an antibody or a fragment thereof.

The term “antibody” as used herein refers to an immunoglobulin molecule comprising an antigen binding site that immuno-specifically binds an antigen. As such, the term antibody encompasses not only whole antibody molecules, but also antibody variants (including derivatives) of antibodies.

In natural antibodies, two heavy chains are linked to each other by disulfide bonds and each heavy chain is linked to a light chain by a disulfide bond. There are two types of light chain, lambda (1) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site.

Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively.

An antigen-binding site, therefore, typically includes six CDRs, comprising the CDR set from each of a heavy and a light chain V region.

Framework Regions (FRs) refer to amino acid sequences interposed between CDRs.

The antibody is preferably a humanized antibody or a human antibody.

As used herein, the term “humanized antibody” refers to an antibody having variable region framework and constant regions from a human antibody, but retaining the CDRs of a previous non-human antibody.

As used herein, the term “human antibody” is intended to include antibodies having variable and constant regions derived from human immunoglobulin sequences. A human antibody may also include amino acid residues not encoded by human immunoglobulin sequences (e.g., mutations introduced by random or site-specific mutagenesis in vitro or by somatic mutation in vivo).

By “fragment of an antibody”, it is herein meant an antigen binding fragment, such as a Fab, a Fab′, a F(ab)′2, a single domain antibody, a ScFv, a Sc(Fv)2, a diabody.

A Fab fragment is a monovalent fragment consisting of the VL, VH, CL and CH1 domains.

A Fab′ fragment is a monovalent fragment consisting of the VL, VH, CL, CH1 domains and hinge region

A F(ab′) 2 fragment is a bivalent fragment comprising two Fab′ fragments linked by a disulfide bridge at the hinge region.

A single domain antibody (sdAb) fragment consists of a VH domain or a VL domain.

Although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (ScFv)).

Divalent and multivalent antibody fragments can form either spontaneously by association of monovalent scFvs, or can be generated by coupling monovalent scFvs by a peptide linker, such as divalent sc(Fv)2. Such single chain antibodies include one or more antigen biding portions or fragments of an antibody.

The antibody as defined above is preferably a monoclonal antibody.

The checkpoint inhibitor as defined above may be selected from the group consisting of a PD-1 antibody, a fragment of a PD-1 antibody, a PD-L1 antibody, a fragment of a PD-L1 antibody, a CD47 antibody, a fragment of a CD47 antibody, a CTLA-4 antibody and a fragment of a CTLA-4 antibody.

The PD-1 inhibitor as defined above is for example a monoclonal anti-PD-1 antibody, such as nivolumab or pembrolizumab, or a fragment thereof.

The PD-L1 inhibitor as defined above is for example an anti-PD-L1 monoclonal antibody, such as atezolizumab, avelumab or durvalumab, or a fragment thereof.

The CD47 inhibitor as defined above is for example an anti-CD47 monoclonal antibody, such as Magrolimab, or a fragment thereof.

The CTLA-4 inhibitor as defined above is for example an anti-CTLA-4 monoclonal antibody, such as ipilimumab, or a fragment thereof.

A preferred checkpoint inhibitor as defined above is a PD-1 inhibitor, more preferably an anti-PD-1 antibody or fragment thereof, such as a monoclonal anti-PD-1 antibody or fragment thereof.

### Pharmaceutical Combination

The present invention particularly relates to a pharmaceutical combination comprising:
(i) at least one nucleic acid comprising an antisense and flipped sequence of a fragment of the sequence coding for a kanamycin resistance protein, as defined above,
(ii) at least one non-immunosuppressive inducer of tumor cell apoptosis, as defined above, and
(iii) at least one checkpoint inhibitor, as defined above.

A preferred pharmaceutical combination as defined above comprises:
(i) at least one nucleic acid comprising an antisense and flipped sequence of a fragment of the sequence coding for a kanamycin resistance protein, wherein said nucleic acid is pVAX14 plasmid of sequence SEQ ID NO: 3,
(ii) at least one non-immunosuppressive inducer of tumor cell apoptosis, wherein said non-immunosuppressive inducer of tumor cell apoptosis is ATRA, arsenic or arsenic trioxide, and
(iii) at least one checkpoint inhibitor, wherein said checkpoint inhibitor is a PD1 inhibitor or a CD47 inhibitor.

In one embodiment, the present invention relates to a pharmaceutical combination as defined above comprising:
(i) only one nucleic acid comprising an antisense and flipped sequence of a fragment of the sequence coding for a kanamycin resistance protein, wherein said nucleic acid is pVAX14 plasmid of sequence SEQ ID NO: 3,
(ii) only one non-immunosuppressive inducer of tumor cell apoptosis, wherein said non-immunosuppressive inducer of tumor cell apoptosis is ATRA, arsenic or arsenic trioxide, and/or
(iii) only one checkpoint inhibitor, wherein said checkpoint inhibitor is a PD1 inhibitor or a CD47 inhibitor.

Said at least one nucleic acid (i), said at least one non-immunosuppressive inducer of tumor cell apoptosis (ii) and said at least one checkpoint inhibitor (iii) of the pharmaceutical combination as defined above, are each an active ingredient, preferably provided in a therapeutically effective amount in the same pharmaceutical composition or in two or more separate pharmaceutical compositions.

The pharmaceutical compositions preferably comprise the active ingredient (said nucleic acid (i), said non-immunosuppressive inducer of tumor cell apoptosis (ii) and/or said checkpoint inhibitor (iii)) and at least one physiologically acceptable carrier.

When the pharmaceutical combination as defined above comprises two or at least two nucleic acids (i) as defined above, said nucleic acids may be provided in the same pharmaceutical composition or in different pharmaceutical compositions.

When the pharmaceutical combination as defined above comprises two or at least two non-immunosuppressive inducers of tumor cell apoptosis (ii) as defined above, said non-immunosuppressive inducers of tumor cell apoptosis may be provided in the same pharmaceutical composition or in different pharmaceutical compositions.

When the pharmaceutical combination as defined above comprises two or at least two checkpoint inhibitors (iii) as defined above, said checkpoint inhibitors may be in the same pharmaceutical composition or in different pharmaceutical compositions.

In one embodiment, said nucleic acid (i), said non-immunosuppressive inducer of tumor cell apoptosis (ii) and said checkpoint inhibitor (iii) of the pharmaceutical combination as defined above are each provided in the form of a separate pharmaceutical composition.

The present invention thus particularly relates to a pharmaceutical combination comprising or consisting of:
(i) a first pharmaceutical composition comprising at least one nucleic acid as defined above and at least one physiologically acceptable carrier,
(ii) a second pharmaceutical composition comprising at least one non-immunosuppressive inducer of tumor cell apoptosis as defined above and at least one physiologically acceptable carrier, and
(iii) a third pharmaceutical composition comprising at least one checkpoint inhibitor as defined above and at least one physiologically acceptable carrier.

In another embodiment, said nucleic acid (i) and said checkpoint inhibitor (iii) may be provided in the same pharmaceutical composition.

The present invention thus also relates to a pharmaceutical combination comprising:
a first pharmaceutical composition comprising:
   at least one nucleic acid comprising an antisense and flipped sequence of a fragment of the sequence coding for a kanamycin resistance protein, as defined above,
   at least one checkpoint inhibitor, as defined above, and
   at least one physiologically acceptable carrier, and
a second pharmaceutical composition comprising at least one non-immunosuppressive inducer of tumor cell apoptosis, as defined above, and at least one physiologically acceptable carrier.

The term “physiologically acceptable carrier” is meant to encompass any carrier, which preferably does not interfere with the effectiveness of the biological activity of the active ingredient and that is preferably not toxic to the host to which is administered. Suitable physiologically acceptable carriers are well known in the art and are described for example in Remington's Pharmaceutical Sciences (Mack Publishing Company, Easton, USA, 1985), which is a standard reference text in this field.

The physiologically acceptable carrier include one or more excipients.

The physiologically acceptable carrier for a pharmaceutical composition as defined above comprising at least one nucleic acid formulated with a nano-vehicle may for example comprise Hepes buffered saline solution (HBSS).

The physiologically acceptable carrier for the non-immunosuppressive inducer of tumor cell apoptosis or for the checkpoint inhibitor may for example comprise a copolymer (such as a nano-vehicle), a liposome and/or a buffer.

A physiologically acceptable carrier used in the pharmaceutical composition comprising said nucleic acid (i) may be a nano-vehicle.

The nucleic acid comprising the antisense and flipped sequence and, optionally, the checkpoint inhibitor can be formulated with a nano-vehicle.

The nano-vehicle is preferably an amphiphilic block copolymer with a tetra arms centered around an ethylidiamine structure.

By “block copolymer”, it is herein meant a polymer comprising at least two sets, or blocks, of polymerized monomeric units. A “block” refers to a motif, obtained by polymerization of a monomer, and which may be repeated within the polymer.

By “amphiphilic block copolymer”, it is herein meant a block copolymer comprising at least one hydrophilic block and at least one hydrophobic block.

Such amphiphilic block copolymers are for example disclosed in documents EP1471944 or EP2819683 and are also referred to as Nanotaxi®.

The nano-vehicle as defined above is for example a tetra-functional amphiphilic block copolymer of formula (I):

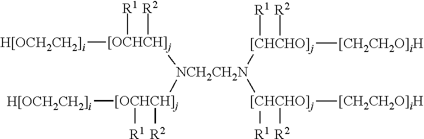

or an organic or mineral salt thereof,
in which i and j represent, independently of one another, an integer of between 1 and 500, and
for each R1, R2 pair, one is hydrogen and the other is a methyl group.

In the formula (I) above, i has preferably a value such that said molecule comprises at least 30% by weight of ethylene oxide units and, preferably, no more than 85% by weight of ethylene oxide units. For example, the compound of formula (I) may comprise between 35% and 50%, preferably around 40% by weight of ethylene oxide units.

In the formula (I) above, i has preferably a value from 10 to 60 and j has preferably a value from 13 to 20. In a preferred embodiment, i is 13 and j is 14.

The mineral salt is preferably an alkali metal salt or an alkaline-earth metal salt. The mineral salt may for example be selected from the group consisting of sodium chloride, potassium chloride, lithium chloride, sodium thiocyanate, calcium chloride (CaCl2) and magnesium chloride (MgCl2).

The mineral salt may be in an isotonic, hypotonic or hypertonic amount.

The nano-vehicle as defined above may be used in a cationic form.

Alternatively, the nano-vehicle as defined above may be a glycosylated tetrafunctional non-ionic block copolymer, wherein said glycosyl moiety is preferably a mannose moiety.

A preferred nano-vehicle is a nano-vehicle as defined above comprising 13 units of ethylene oxide and 14 units of propylene oxide centered around an ethylenediamine. Such a preferred nano-vehicle has the following formula (II)

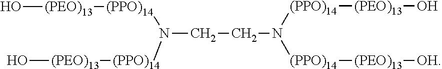

In the above formula (II), “PEO” means polyethylene oxide and “PPO” means polypropylene oxide.

Formulation of said nucleic acid (i) in a nano-vehicle as defined above may be obtained by equivolumetric mixing of an aqueous solution of said nucleic acid, in particular at the desired concentration, with a solution comprising the nano-vehicle.

The pH of the solution comprising the nano-vehicle is preferably comprised from 6.5 to 8, more preferably from 7 to 7.8, for example 7.4.

The solution comprising the nano-vehicle preferably comprises a buffer, such as Tyrode's medium.

Tyrode's medium comprises 3 mM CaCl2, 2 mM MgCl2, 6 mM KCl, 140 mM NaCl, 10 mM glucose and 10 mM Hepes, pH 7.4.

Since the mixing of both solutions is equivolumetric, it is preferred that the aqueous solution comprising said nucleic acid is at 2× concentration and that the solution comprising the nano-vehicle and the buffer is also at 2× concentration, so as to get a final solution at 1× concentration.

When both said nucleic acid (i) and said checkpoint inhibitor (ii) are formulated with a nano-vehicle as defined above, a mixture is first made between the nano-vehicle and the nucleic acid (i), for example as described above, and then the checkpoint inhibitor is added.

The pharmaceutical combination as defined above may for example corresponds to a kit. The kit may further comprise instructions for the use in the treatment of cancer.

Alternatively, the first, second and/or third pharmaceutical compositions may be commercialized separately.

The pharmaceutical composition as defined above comprising said nucleic acid (i) preferably corresponds to vaccine composition.

The pharmaceutical composition as defined above comprising said nucleic acid (i) preferably comprises from 0.5 mg to 10 mg of nucleic acid, preferably from 0.5 mg to 2 mg of nucleic acid.

The pharmaceutical composition as defined above comprising said non-immunosuppressive inducer of tumor cell apoptosis (ii) preferably comprises from 5 mg to 500 mg of non-immunosuppressive inducer of tumor cell apoptosis, preferably from 10 mg to 200 mg of non-immunosuppressive inducer of tumor cell apoptosis, for example from 10 mg to 100 mg of non-immunosuppressive inducer of tumor cell apoptosis.

The pharmaceutical composition as defined above comprising ATRA (ii) preferably comprises from 5 mg to 100 mg of ATRA, preferably from 5 mg to 50 mg of ATRA, for example from 10 mg to 30 mg of ATRA.

The pharmaceutical composition as defined above comprising azacytidine preferably comprises from 5 mg/ml to 100 mg/ml of azacytidine, more preferably from 10 mg/ml to 50 mg/ml of azacytidine, for example from 20 mg/ml to 30 mg/ml of azacytidine.

The pharmaceutical composition as defined above comprising arsenic or arsenic trioxide (ii) preferably comprises from 0.2 to 20 mg/ml of arsenic or arsenic trioxide, more preferably from 0.5 mg/ml to 10 mg/ml of arsenic or arsenic trioxide, for example from 1 mg/ml to 5 mg/ml of arsenic or arsenic trioxide.

The pharmaceutical composition as defined above comprising said checkpoint inhibitor (iii) preferably comprises from 1 mg to 500 mg of said checkpoint inhibitor, more preferably from 5 mg to 200 mg of said checkpoint inhibitor, for example from 10 mg to 100 mg of said checkpoint inhibitor.

The pharmaceutical composition as defined above comprising said checkpoint inhibitor (iii) may comprise from 0.1 mg/ml to 200 mg/ml of said checkpoint inhibitor, more preferably from 1 mg/ml to 100 mg/ml of said checkpoint inhibitor, for example from 10 mg/ml to 50 mg/ml of said checkpoint inhibitor.

Each of said nucleic acid (i), said non-immunosuppressive inducer of tumor cell apoptosis (ii) and said checkpoint inhibitor (iii) may be provided in the form of one or more dosage units.

The term “dosage unit” herein refers to a physically discrete unit suitable as unitary dosage, each unit containing a predetermined quantity of active ingredient calculated to produce the desired therapeutic effect.

The pharmaceutical combination as defined above may thus comprise:
(i) one or more dosage units of a first pharmaceutical composition as defined above, wherein said first pharmaceutical composition comprises at least one nucleic acid comprising an antisense and flipped sequence of a fragment of the sequence coding for a kanamycin resistance protein and at least one pharmaceutical acceptable carrier,
(ii) one or more dosage units of a second pharmaceutical composition as defined above, wherein said second pharmaceutical composition comprises at least one non-immunosuppressive inducer of tumor cell apoptosis and at least one pharmaceutical acceptable carrier, and
(iii) one or more dosage units of a third pharmaceutical composition as defined above, wherein said third pharmaceutical composition comprises at least one checkpoint inhibitor and at least one pharmaceutical acceptable carrier.

Alternatively, the pharmaceutical combination may comprise:
(i) one or more dosage units of a first pharmaceutical composition as defined above, wherein said first pharmaceutical composition comprises:
   a. at least one nucleic acid comprising an antisense and flipped sequence of a fragment of the sequence coding for a kanamycin resistance protein,
   b. at least one checkpoint inhibitor, and
   c. at least one pharmaceutical acceptable carrier, and
(ii) one or more dosage units of a second pharmaceutical composition as defined above, wherein said second pharmaceutical composition comprises at least one non-immunosuppressive inducer of tumor cell apoptosis and at least one pharmaceutical acceptable carrier.

The pharmaceutical combination as defined above may comprise at least one other drug, preferably suitable for the treatment of cancer.

By “other drug” or “another drug”, it is herein meant a drug, which is not a nucleic acid comprising an antisense and flipped sequence of a fragment of the sequence coding for a kanamycin resistance protein, which is not a checkpoint inhibitor, and which is not a non-immunosuppressive inducer of tumor cell apoptosis.

Said other drug is preferably an anti-cancer drug, such as decitabine.

In another embodiment, the pharmaceutical combination as defined above does not comprise any other drug suitable for the treatment of cancer and, preferably, does not comprise any other drug.

### Therapeutic Use

The present invention particularly relates to the pharmaceutical combination as defined above for use as a medicament, in particular for the treatment of cancer.

The present invention particularly relates to a method of treatment of cancer, comprising administering to a subject in need thereof the pharmaceutical combination as defined above.

Administration of the pharmaceutical combination as defined above advantageously allows reducing tumor burden and increasing memory T-cells, thereby prolonging remission, preventing relapse and resulting in a durable protection against cancer.

By “treatment of cancer “, it is herein meant a therapeutic use (i.e. on a patient having cancer). The term “treatment” not only includes a treatment leading to complete cure of cancer, but also a treatment slowing down the progression of cancer and/or prolonging the survival of the patient. It may also be a treatment slowing down the progression of cancer initiation ie as in preneoplasia for example myelodysplastic syndromes.

As used herein, the term “cancer” refers to any type of malignant (i.e. non benign) tumor.

Cancer may be a solid tumor, preferably selected from the group consisting of a carcinoma, an adenocarcinoma, a sarcoma, a melanoma, a mesothelioma and a blastoma.

Cancer is preferably breast cancer.

Cancer may also be a blood cancer, preferably selected from the group consisting of lymphoid leukaemia, myeloid leukaemia, acute promyelocytic leukaemia (APL), myelodysplastic syndrome (MDS), acute myeloid leukaemia (AML), myelomonocytic leukaemia (CMML), chronic lymphocytic leukaemia (CLL), chronic myelogenous leukaemia (CML), childhood acute lymphoblastic leukaemia (ALL), Hodgkin lymphoma (HL), non-Hodgkin lymphoma (NHL) and multiple myeloma (MM) or preneoplasia such as monoclonal gammopathy of undetermined significance (MGUS), which serves as a precursor to myeloma (MM)

By “subject in need thereof”, it is herein meant a subject suffering from or susceptible of suffering from the disease to be treated. The subject to be treated in the frame of the invention is preferably a human being. However, the veterinary use of the pharmaceutical combination is also contemplated by the present invention. The subject may thus be a human being or a non-human mammal.

Said at least one nucleic acid (i), said non-immunosuppressive inducer of tumor cell apoptosis, (ii) and said at least one checkpoint inhibitor (iii) are administered in a therapeutically effective amount.

By “therapeutically effective amount”, it is herein meant an amount sufficient to achieve a concentration of active ingredient, which is capable of preventing, treating or slowing down the disease to be treated. Such concentrations can be routinely determined by those of skilled in the art. The amount of the active ingredient actually administered will typically be determined by a physician, in the light of the relevant circumstances, including the condition to be treated, the chosen route of administration, the actual active ingredient administered, the age, weight, and response of the individual subject, the severity of the subject's symptoms, and the like.

Said nucleic acid (i), said non-immunosuppressive inducer of tumor cell apoptosis (ii) and/or said checkpoint inhibitor (iii) may be administered simultaneously, i.e. at the same time, or sequentially, i.e. at different times during the course of a common treatment schedule. Said simultaneous or sequential administration may change during the course of treatment.

For example, said nucleic acid (i), said non-immunosuppressive inducer of tumor cell apoptosis (ii) and/or said checkpoint inhibitor (iii) may be administered at the same time at the beginning of the treatment, and sequentially in the following.

In one embodiment, said nucleic acid (i), said non-immunosuppressive inducer of tumor cell apoptosis (ii) and/or said checkpoint inhibitor (iii) are administered separately at the same time at the beginning of the treatment, and sequentially in the following.

The nucleic acid as defined above may be administered as a single dose, once a week, once every two weeks, once every three weeks or once a month, for example for at least 8 weeks, preferably for at least 3 months, more preferably for at least 4 months.

The nucleic acid as defined above may for example be administered once to four times or at least four times, with an interval of two weeks between two administrations, optionally followed by one or at least two administrations at an interval of one to two months between two administrations.

The non-immunosuppressive inducer of tumor cell apoptosis, in particular ATRA, as defined above, may be administered once a day, for example for ten days, preferably at the beginning of the treatment, more preferably each time said nucleic acid (i) is administered.

The checkpoint inhibitor as defined above may be administered at a single dose, for example twice a week, once a week, once every two weeks, once every three weeks or once a month, for example for at least 8 weeks, preferably for at least 3 months, more preferably for at least 4 months.

The checkpoint inhibitor (iii) may be administered at the same time as said nucleic acid (i), in particular when the checkpoint inhibitor and said nucleic acid (i) are comprised in the same pharmaceutical composition.

Frequency of administration of active ingredient (i), (ii) and/or (iii) may vary during the course of treatment. For example, frequencies may be shorter at the beginning of the treatment and then more spaced out during the course of treatment.

The pharmaceutical combination is preferably administered at least until disappearance of the tumor(s), more preferably until at least one month after disappearance of the tumor(s).

The pharmaceutical combination may also be administered in a stable state of disease or during complete remission, to prevent re-occurrence of the tumor(s).

In one embodiment,
the nucleic acid (i) as defined above is administered once or at least twice (for example from two to four times) with an interval of two weeks between two administrations, optionally followed by one or at least two administrations at an interval of one to two months,
the non-immunosuppressive inducer of tumor cell apoptosis (ii) is preferably ATRA and is administered orally once a day for 10 days each time said nucleic acid (i) is administered, and
the checkpoint inhibitor (iii) as defined above is administered every two weeks, preferably for at least 8 weeks, or each time said nucleic acid (i) is administered when the checkpoint inhibitor and said nucleic acid (i) are comprised in the same pharmaceutical composition.

The amount of nucleic acid (i) to be administered depends, e.g., on the strength of the promoter used, the condition of the mammal intended for administration (e.g., weight or age), the route of administration and the type of formulation.

The nucleic acid as defined above may be administered at a dosage of from 1 μg to 8 mg, preferably from 100 μg to 5 mg, more preferably from 500 μg to 2 mg, in particular for a human adult.

The nucleic acid as defined above may for example be administered at a dosage ranging from 500 μg at the beginning of the treatment to 2 mg at the end of the treatment, in particular for a human adult.

The amount of non-immunosuppressive inducer of tumor cell apoptosis (ii) to be administered depends, e.g. on the condition of the mammal intended for administration (e.g., weight or age), the route of administration, the type of non-immunosuppressive inducer of tumor cell apoptosis and the type of formulation.

The non-immunosuppressive inducer of tumor cell apoptosis may be administered at a dosage of from 20 mg/m² to 150 mg/m², preferably from 30 mg/m² to 100 mg/m², for example from 40 mg/m² to 75 mg/m², preferably once a day for 7 to 10 days, preferably every time said nucleic acid is administered.

The non-immunosuppressive inducer of tumor cell apoptosis ATRA may for example be administered at a dosage of 45 mg/m², preferably once a day for 10 days, preferably every time said nucleic acid is administered.

The non-immunosuppressive inducer of tumor cell apoptosis arsenic trioxide may be administered at a dosage of 0.15 mg/kg, preferably once a day for 10 days, every time said nucleic acid is administered.

The non-immunosuppressive inducer of tumor cell apoptosis azacytidine may be administered at a dosage of 0.15 mg/kg, preferably once a day for 10 days, every time said nucleic acid is administered.

The amount of checkpoint inhibitor (iii) to be administered depends, e.g. on the condition of the mammal intended for administration (e.g., weight or age), the route of administration, the type of inhibitor and the type of formulation.

The checkpoint inhibitor (iii) as defined above may be administered at a dosage of from 0.5 mg/kg to 20 mg/kg, preferably from 1 mg/kg to 10 mg/kg, more preferably from 1.3 mg/kg to 10 mg/kg.

Said nucleic acid (i), said non-immunosuppressive inducer of tumor cell apoptosis (ii) and/or said checkpoint inhibitor (iii) may be administered via an intradermal, intra-epidermal, subcutaneous, intramuscular, intravenous, oral route or intra-tumoral route.

The nucleic acid (i) as defined above may be administered in a naked form, i.e. free from any delivery vehicles. To this end, the nucleic acid is simply diluted in a physiologically acceptable solution, such as sterile saline or sterile buffered saline, with or without a carrier. When present, the carrier is preferably isotonic, hypotonic, or weakly hypertonic, and has a relatively low ionic strength, such as provided by a sucrose solution, e.g., a solution containing 20% sucrose.

Alternatively, the nucleic acid (i) as defined above may be administered in association with agents that assist in cellular uptake. Examples of such agents are (i) chemicals that modify cellular permeability, such as bupivacaine (see, e.g., WO 94/16737), (ii) liposomes or viral particles for encapsulation of the nucleic acid, (iii) cationic lipids, (iv) silica, gold, or tungsten microparticles, which associate themselves with the nucleic acid or (v) a nano-vehicle, such as nanotaxi®.

Anionic and neutral liposomes are well-known in the art (see, e. g., Liposomes: A Practical Approach, RPC New Ed, IRL press, 1990), for a detailed description of methods for making liposomes) and are useful for delivering a large range of products, including polynucleotides.

Cationic lipids are also known in the art and are commonly used for gene delivery. Such lipids include Lipofectin Tm also known as DOTMA (N-[I-(2,3-dioleyloxy) propyls N, N, N-trimethylammonium chloride), DOTAP (1, 2-bis(oleyloxy)-3 (trimethylammonio) propane), DDAB (dimethyldioctadecyl-ammonium bromide), DOGS (dioctadecylamidologlycyl spermine) and cholesterol derivatives such as DC-Chol (3 beta-(N—(N′, N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. Cationic lipids for gene delivery are preferably used in association with a neutral lipid such as DOPE (dioleyl phosphatidylethanolamine), as described in WO 90/11092 as an example.

Formulation containing cationic liposomes may optionally contain other transfection-facilitating compounds. A number of them are described in WO 93/18759, WO 93/19768, WO 94/25608, and WO 95/02397. They include spermine derivatives useful for facilitating the transport of DNA through the nuclear membrane (see, for example, WO 93/18759) and membrane permeabilizing compounds such as GALA, Gramicidine S, and cationic bile salts (see, for example, WO 93/19768).

Gold or tungsten microparticles may be used for gene delivery, as described in WO 91/00359 or WO 93/17706. The microparticle-coated polynucleotide is injected via intradermal or intraepidermal routes using a needleless injection device (“gene gun”), such as those described in U.S. Pat. Nos. 4,945,050, 5,015,580, and WO 94/24263.

In a preferred embodiment, the nucleic acid is formulated with a nano-vehicle, such as nanotaxi®, optionally with the checkpoint inhibitor, in particular as defined above.

The route of administration for the nucleic acid (i) as defined above is any conventional route used in the field of DNA vaccination.

As general guidance, a nucleic acid of the invention may be administered via a parenteral route, e.g., by an intradermal, intraepidermal, or intramuscular route.

The choice of administration route depends on the formulation that is selected. A polynucleotide formulated in association with bupivacaine is advantageously administered into muscles.

When a neutral or anionic liposome or a cationic lipid, such as DOTMA or DC-Chol, is used, the formulation can be advantageously injected via intramuscular or intradermal routes.

A nucleic acid in a naked form can advantageously be administered via the intramuscular, intradermal, or subcutaneous routes. In addition, electroporation can be developed to improve delivery of DNA to muscle (Mir et al. 1999, Proc Natl Acad Sci USA. 96:4262-7).

A nucleic acid formulated in a nano-vehicle as defined above, such as nanotaxi®, is preferably administered via intramuscular route. For HR-MDS (Higher-Risk Myelodysplastic Syndromes) patients, who have coagulation problems, the intradermal route is preferably used.

The route of administration for the non-immunosuppressive inducer of tumor cell apoptosis is preferably oral route, in particular for ATRA, intravenous route, in particular for ATO or azacytidine, or subcutaneously route for azacytidine.

The route of administration for the checkpoint inhibitor is preferably intravenous route.

The present invention also relates to a pharmaceutical combination for use as defined above, in combination with at least one other drug, preferably suitable for the treatment of cancer.

The other drug is particularly as defined above.

In another embodiment, the pharmaceutical combination as defined above is not used in combination with at least one other drug suitable for the treatment of cancer.

All references cited herein, including journal articles or abstracts, published patent applications, issued patents or any other references, are entirely incorporated by reference herein, including all data, tables, figures and text presented in the cited references.

The invention will be further illustrated in the following examples and figures.

### BRIEF DESCRIPTION OF THE SEQUENCES

SEQ ID NO: 1 corresponds to an antisense and flipped sequence of a fragment of the sequence coding for the kanamycin resistance protein.

SEQ ID NO: 2 corresponds to the flipper region consisting of an antisense and flipped sequence of a fragment of the sequence coding for the kanamycin resistance protein and of an antisense and flipped sequence of a fragment of the pVAX1 plasmid.

SEQ ID NO: 3 corresponds to the complete sequence of the pVAX14 plasmid comprising the flipper region of sequence SEQ ID NO: 2.

SEQ ID NO: 4 corresponds to the sequence of the immunogenic polypeptide encoded by ORF1.

SEQ ID NO: 5 corresponds to the sequence of the immunogenic polypeptide encoded by ORF2.

SEQ ID NO: 6 corresponds to the sequence of the immunogenic polypeptide encoded by ORF5.

SEQ ID NO: 7 corresponds to the sequence of pVAX1 plasmid.

### BRIEF DESCRIPTION OF THE DRAWINGS

FIGS. 1 and 2: pVAX14 delays tumor development, slowing down cancer initiation and progression, increases survival and decreases tumor burden in a mouse model of triple negative breast cancer. The experiment was terminated on day 85.

FIG. 3: Experimental schedule for anti-PD-1+ATRA+pVX14 for the breast cancer mouse model.

FIG. 4: Expression of PD-1 showing anti-PD-1 antibody (PD-1) hitting its target on day 38.

FIG. 5: Expression of Memory T-cells (CD44 hi/CD62Llo in CD4+ cells) (Tmem) on day 53.

FIG. 6: Challenge of long-term survivors (>200 days) show the mice treated with the combination of anti-PD-1 antibody+ATRA+pVAX14 may be protected against tumor challenge. Long-term survivors challenged with breast cancer cells (106) did not develop tumors, whereas the control mice developed tumors readily within 6 days.

### EXAMPLES

### Example 1: Combined Use of pVAX14, ATRA and Anti-PD-1 Antibody Induces Reduced Tumor Burden and Durable Protection in Breast Cancers

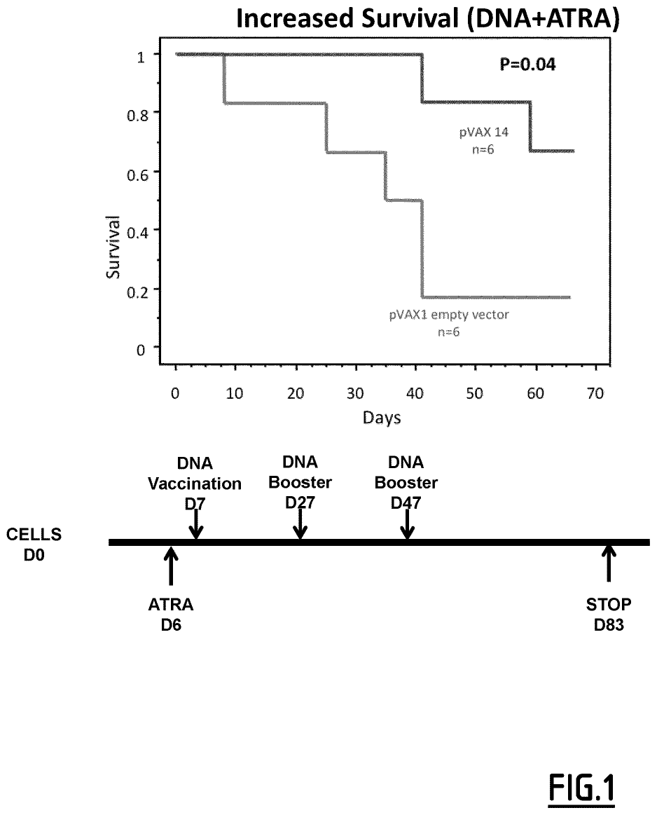

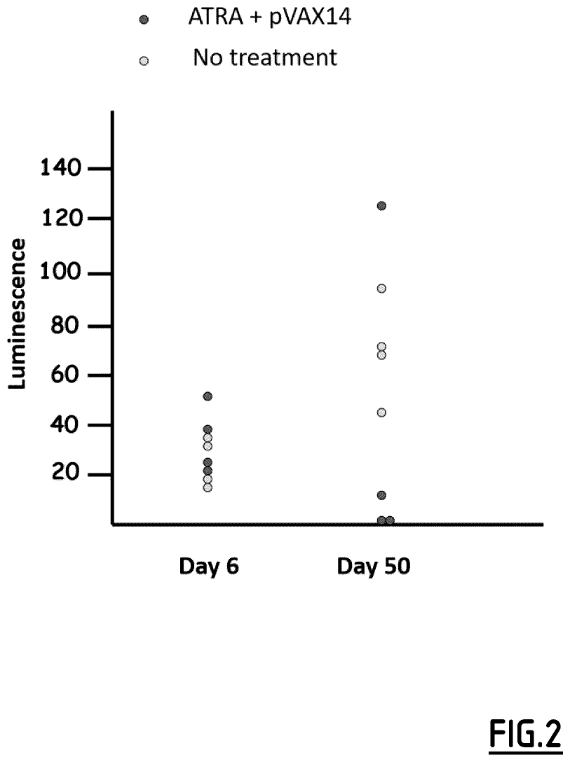

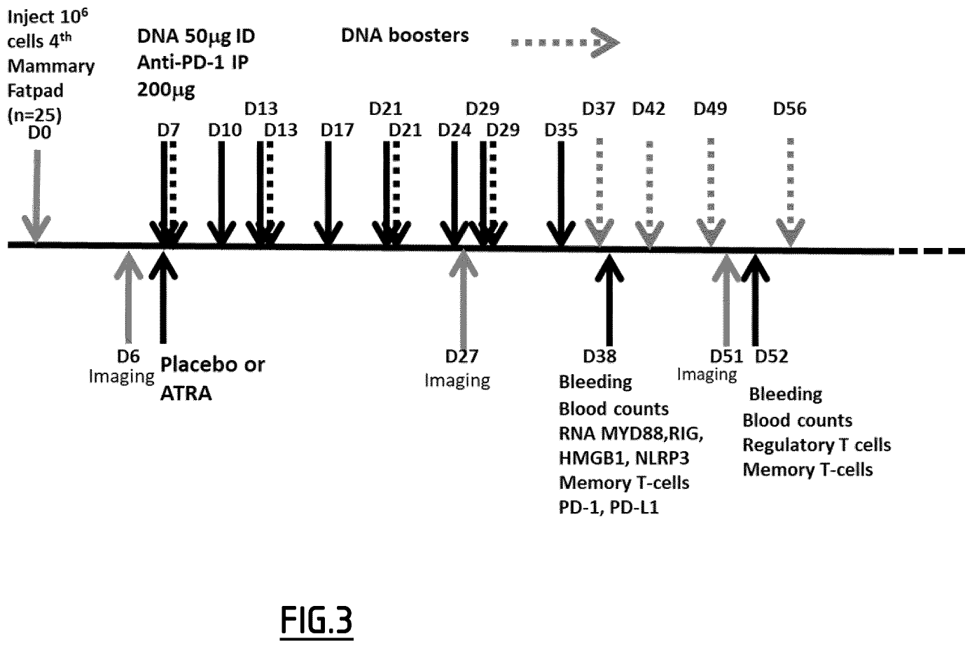

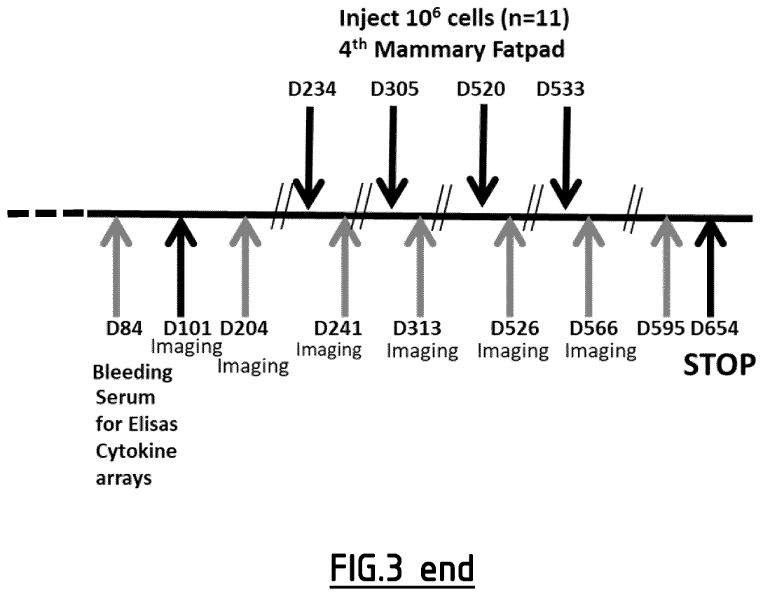

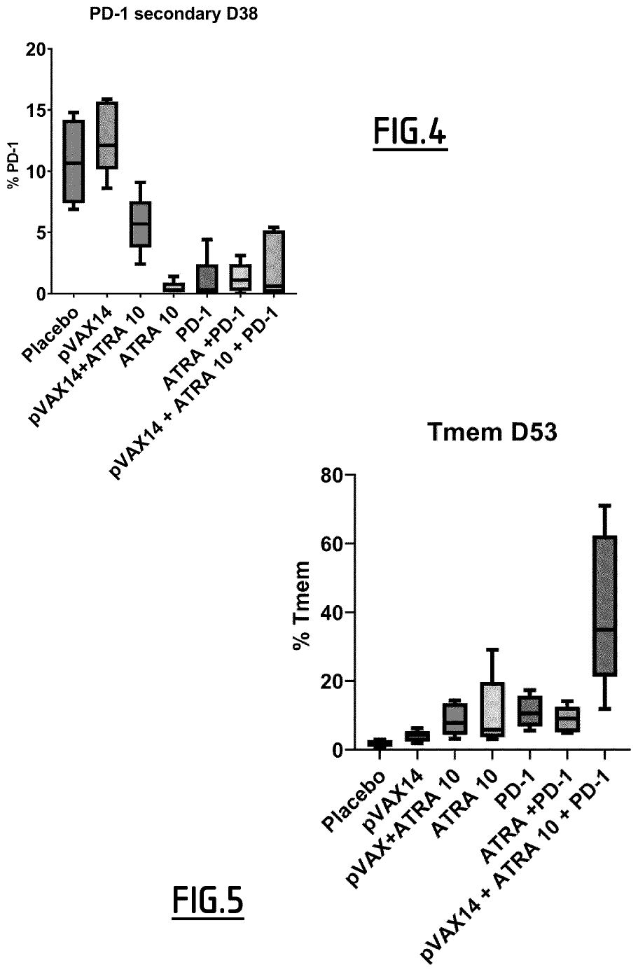

Studies on a breast cancer model using the 4T1 derived cell line (Tao et al, 2008) containing the luciferase gene show that the combination of ATRA+pVAX14 extends the lifespan of the transplanted mice compared to the parental vector pVAX1 with a reduction in tumour burden as assayed by luciferase imaging (FIGS. 1 and 2). Addition of immunotherapy of pVAX14+ATRA with the immune checkpoint inhibitor anti-PD-1 antibody has been undertaken to test the hypothesis that this combination will induce durable remissions by increasing memory T-cells. The protocol is illustrated in FIG. 3. It was observed a reduction in PD-1 levels measured by flow cytometry, showing that the antibody hits its target (FIG. 4) and increased memory T-cells with the highest levels in mice treated with the combination of pVAX14+ATRA+anti-PD-1 antibody (FIG. 5).

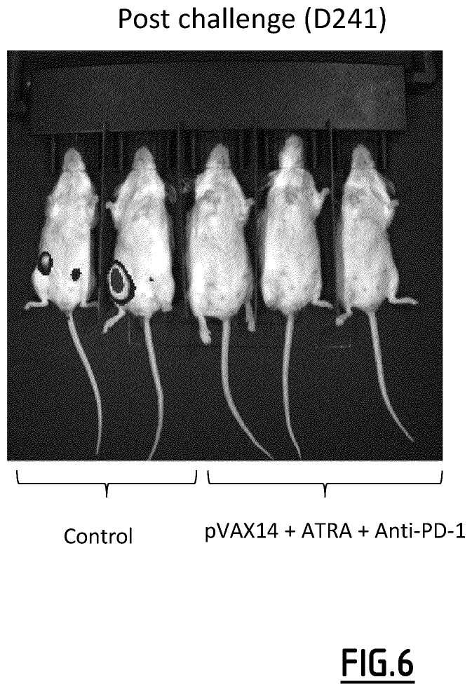

The long-term survivors have been followed-up and challenged with the 4T1 cells at intervals 4 times until the mice started dying without disease at 21.5 months after the start of the experiment. The long-term survivors had no evidence of tumors (FIG. 6). Experiment was thus terminated at day 654 as the mice were dying (mice normally live 24 months), in the absence of detectable disease, as imaged on day 595 (data not shown). The ATRA+DNA combination thus activates the immune system and combined with other strategies, such as an anti-PD-1 ligand, prolongs lifespan and induces durable remissions to prevent relapse.

As a conclusion, the addition of pVAX14 and ATRA to anti-PD-1 antibody therapy induces reduced tumor burden and durable protection in breast cancers. This strategy is expected to have broader applications to other cancers and to be a general principle for other checkpoint inhibitors.

### REFERENCES

1. Bagchi S, Yuan R, Engleman E G. Immune Checkpoint Inhibitors for the Treatment of Cancer: Clinical Impact and Mechanisms of Response and Resistance. Annu Rev Pathol. January 24; 16:223-249. 2021. doi: 10.1146/annurev-pathol-42020-042741. Epub 2020 Nov. 16.

PMID: 33197221 Review.
2. Furugaki K, Pokorna K, Le Pogam C, Aoki M, Reboul M, Bajzik V, Krief P, Janin A, Noguera M E, West R, Charron D, Chomienne C, Pla M, Moins-Teisserenc H, Padua R A. DNA vaccination with all-trans retinoic acid treatment induces long-term survival and elicits specific immune responses requiring CD4+ and CD8+ T-cell activation in an acute promyelocytic leukemia mouse model. Blood 15:653-6, 2010. Epub 2009 Nov. 19. PMID: 19965687
3. Le Pogam C, Patel S, Gorombei P, Guerenne L, Krief P, Bernasconi E, Tekin N, Sicre F, Schlageter M-E, Chopin M, Mathews V, West R R, PLA M, Fenaux P, Chomienne C, Padua R A. DNA-mediated immunotherapy induces immune responses and extends life-span in two different mouse models of myeloid malignancies. Oncotarget, 6:32494-508, 2015 PMID: 26378812
4. Omidvar N, Kogan S, Beurlet S, Le Pogam C, Janin A, West R, Noguera M E, Reboul M., Soulie A., Le Boeuf C, Setterblad N, Felsher D, Lagasse, E, Mohamedali A, Thomas N S, Fenaux P, Fountenay M, Pla M, Mufti G J, Weissmann I, Chomienne C, Padua R A. BCL-2 and mutant NRAS interact physically and functionally in a mouse progressive myelodysplasia. Cancer Research 67:11657-67, 2007. PMID: 18089795
5. Padua R A, Larghero J, Robin M, le Pogam C, Schlageter M-H, Muszlak S, Fric J, West R, Rousselot P, Phan T H, Mudder L, Triesserenc H, Carpentier A, Kogan S, Degos L, Pla M, Bishop M, Stevenson F K, Charron D, Chomienne C. PML-RARA-targeted DNA vaccine induces protective immunity in a mouse model of leukemia. Nature Medicine 9:1413-1417, 2003.

PMID: 14566333
6. Patel S, Guerenne L, Gorombei P, Omidvar N, Schlageter M, Alex A A, Ganesan S, West R, Ades L, Mathews V, Krief P, Pla M, Fenaux P, Chomienne C, Padua R A. PVAX14-mediated add-on immunotherapy combined with arsenic trioxide and all-trans retinoic acid (ATO+ATRA) targeted therapy effectively increases survival of APL mice. Blood Cancer Journal, December 11; 5: e374. doi: 10.1038/bcj.2015.102 PMID: 26657197
7. Pokorna K, Chopin M, Le Pogam C, Balitrand N, Reboul M, Cassinat B, Chomienne C, Padua R A, Pla M. Tracking of oncogene positive cells in preclinical models: biomarker of long term drug efficacy. Molecular and Cellular Probes 27:1-5, 2013. PMID: 22906630
8. Robin M, Andreu-Gallien J, Schlageter M-H, Bengoufa D, Guillemot I, Pokorna K, Robert C, Larghero J, Rousselot P, Raffoux E, Dombret H, Fenaux P, Pla M, Charron D, Padua R A, Chomienne C. Frequent antibody production against RAR□ in both APL mice And patients. Blood 108:1972-1974, 2006. PMID: 16728697
9. Tao K, Fang M, Alroy J, Sahagian G G. Imagable 4T1 model for the study of late stage breast cancer. BMC Cancer August 9; 8:228. doi: 10.1186/1471-2407-8-228, 2008. PMID: 18691423

1. A pharmaceutical combination comprising:
(i) at least one nucleic acid comprising an antisense and flipped sequence of a fragment of the sequence coding for a kanamycin resistance protein, wherein said nucleic acid is capable of eliciting an immune response in a mammal,
(ii) at least one non-immunosuppressive inducer of tumor cell apoptosis-, and
(iii) at least one checkpoint inhibitor.

2. The pharmaceutical combination according to claim 1, wherein said nucleic acid comprises a sequence selected from the group consisting of:
a) sequence SEQ ID NO: 2,
b) a sequence at least 80% identical to SEQ ID NO: 2,
c) a fragment of at least 50 consecutive nucleotides of SEQ ID NO: 2, and
d) sequence SEQ ID NO: 3.

3. The pharmaceutical combination according to claim 1, wherein said antisense and flipped sequence encodes at least one immunogenic peptide selected from the group consisting of sequence SEQ ID NO: 4 and SEQ ID NO: 5.

4. The pharmaceutical combination according to claim 1, wherein said checkpoint inhibitor is selected from the group consisting of a PD-1 inhibitor, a PD-L1 inhibitor, a CD47 inhibitor and a CTLA-4 inhibitor.

5. The pharmaceutical combination according to claim 1, wherein said non-immunosuppressive inducer of tumor cell apoptosis is ATRA, arsenic, arsenic trioxide or azacytidine.

6. The pharmaceutical combination according to claim 1, wherein said nucleic acid (i) is formulated with a nano-vehicle.

7. The pharmaceutical combination according to claim 1, wherein each of said nucleic acid (i), said non-immunosuppressive inducer of tumor cell apoptosis (ii) and said checkpoint inhibitor (iii) is provided in the form of a separate pharmaceutical composition.

8. The pharmaceutical combination according to claim 1, wherein said nucleic acid (i) and said checkpoint inhibitor (iii) are provided in the same pharmaceutical composition.

9. The pharmaceutical combination according to claim 1, wherein said nucleic acid (i), said non-immunosuppressive inducer of tumor cell apoptosis (ii) and said checkpoint inhibitor (iii) are provided in the form of one or more doses of a pharmaceutical composition.

10. A method of treatment of cancer comprising administering to a subject in need thereof the pharmaceutical combination according to claim 1.

11. The method according to claim 10, wherein said nucleic acid (i), said non-immunosuppressive inducer of tumor cell apoptosis (ii) and/or said checkpoint inhibitor (iii) are administered simultaneously or sequentially.

12. The method according to claim 10, wherein said nucleic acid (i), said non-immunosuppressive inducer of tumor cell apoptosis (ii) and/or said checkpoint inhibitor (iii) is/are administered by intradermal, intra-epidermal, subcutaneous, intramuscular, intravenous, oral route or intratumoral route.

13. The method according to claim 10, wherein
said nucleic acid (i) is administered at a dosage of from 500 μg to 2 mg,
said non-immunosuppressive inducer of tumor cell apoptosis (ii) is ATRA and is administered at a dosage of 45 mg/m² and/or
said checkpoint inhibitor (iii) is administered at a dosage of from 1 to 10 mg/kg.

14. The method according to claim 10, wherein:
said nucleic acid (i) is administered once or at least twice, preferably with an interval of two weeks between two administrations,
said non-immunosuppressive inducer of tumor cell apoptosis (ii) is administered once a day for 10 days, each time said nucleic acid (i) is administered, and/or
said checkpoint inhibitor (iii) is administered each time said nucleic acid (i) is administered.

15. The method according to claim 10, wherein said cancer is a solid tumor, preferably breast cancer, or a blood cancer.


SEQUENCE LISTING (7 sequences)

SEQ ID NO: 1 - DNA, 472 bp, synthetic construct
  source 1..472: mol_type = other DNA
  misc_feature 1..472: note = Antisense and flipped sequence of a fragment of the sequence coding for the kanamycin resistance protein
gatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcat

SEQ ID NO: 2 - DNA, 825 bp, synthetic construct
  source 1..825: mol_type = other DNA
  misc_feature 1..825: note = Flipper region
  misc_feature 1..472: note = Antisense and flipped sequence of a fragment of the sequence coding for the kanamycin resistance protein
  misc_feature 473..825: note = Antisense and flipped sequence of a fragment of the pVax1 plasmid
gatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcac

SEQ ID NO: 3 - DNA, 3540 bp, synthetic construct
  source 1..3540: mol_type = other DNA
  misc_feature 1..3540: note = pVax14
  misc_feature 763..1587: note = Flipper region
  misc_feature 763..1234: note = Antisense and flipped sequence of a fragment of the sequence coding for the kanamycin resistance protein
  misc_feature 1235..1587: note = Antisense and flipped sequence of a fragment of pVax1
  misc_feature 1816..2610: note = Sequence coding for the kanamycin resistance protein
tgctggccttttgctcacatgttcttgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatgaaaaacgccagcaacgcggc

SEQ ID NO: 4 - protein, 35 aa, synthetic construct
  REGION 1..35: note = Immunogenic polypeptide
MRCFAWWSNGQVAGSSVCSRRIASAMMDTFSAGAR

SEQ ID NO: 5 - protein, 35 aa, synthetic construct
  REGION 1..35: note = Immunogenic polypeptide
MFRLVVEWAGSRIKRMQPPHCISHDGYFLGRSKVR

SEQ ID NO: 6 - protein, 59 aa, synthetic construct
  REGION 1..59: note = Immunogenic polypeptide
MPAIVFPILPLAVLPHPTPQNRMTPTQTMRCNFLILLGKDSGSGTFQGQGRHGGGLGRQ

SEQ ID NO: 7 - DNA, 2999 bp, synthetic construct
  misc_feature 1..2999: note = pVAX1 vector
  regulatory 137..724: note = promoter - CMV promoter; regulatory_class = promoter
  regulatory 664..683: note = promoter - T7 promoter/priming site; regulatory_class = promoter
  misc_feature 696..811: note = Multiple cloning site
  misc_feature 1226..2020: note = sequence coding for the kanamycin resistance protein
  rep_origin 2320..2993: note = pUC origin
  source 1..2999: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt